(19) United States
(12) Patent Application Publication
(10) Pub. No.: US 2003/0119022 A1
(43) Pub. Date: Jun. 26, 2003

(54) Recognition and cleavage at the DNA major groove

(76) Inventors: Edward B. Skibo, Mesa, AZ (US); Chengguo Xing, Somerville, MA (US)

Correspondence Address: FENNEMORE CRAIG, 3003 NORTH CENTRAL AVENUE, SUITE 2600, PHOENIX, AZ, 85012, US

(21) Appl. No.: 10/198,333

(22) Filed: Jul. 18, 2002

Related U.S. Application Data
(60) Provisional application No. 60/306,360, filed on Jul. 18, 2001.

(51) Int. Cl. C12Q 1/68

(52) U.S. Cl. 435/6

(57) ABSTRACT

DNA recognition agents based on the indole-based aziridinyl eneimine and the cyclopent[b]indole methide species are described. The recognition process involved either selective alkylation or intercalating interactions in the major groove. DNA cleavage resulted from phosphate backbone alkylation (hydrolytic cleavage) and N(7)-alkylation (piperidine cleavage). The formation and fate of the eneimine was studied using enriched ¹³C NMR spectra and x-ray crystallography. The aziridinyl eneimine specifically alkylates the N(7) position of DNA resulting in direction of the aziridinyl alkylating center to either the 3′- or 5′-phosphate of the alkylated base. The eneimine species forms dimers and trimers that appear to recognize DNA at up to three base pairs. The cyclopent[b]indole quinone methide recognizes the 3′-GT-5′ sequence and alkylates the guanine N(7) and the thymine 6-carbonyl oxygen causing the hydrolytic removal of these bases. New classes of DNA recognition agents have been developed and the utility of ¹³C-enrichment and ¹³C-NMR to study DNA alkylation reactions is disclosed.

### TECHNICAL FIELD

This application claims the benefit of U.S. Provisional Application No. 60/306,360, filed Jul. 18, 2001.

Financial assistance for this project was provided by the U.S. government through the National Science Foundation under Grant Number CHE-9522640, through the National Institutes of Health under Grant Number CA 73758, and through the Arizona Disease Control Commission. The U.S. Government may own certain rights to this invention.

### BACKGROUND OF THE INVENTION

The recognition of DNA sequences and subsequent cleavage by chemical nucleases have been the subject of intense interest. The pyrrolopeptides, such as distamycin and netropsin, recognize DNA by binding to the minor groove. See Wemmer, D. E. and Dervan, P. B., Targeting the Minor Groove of DNA, Curr. Opin. Struct. Biol. 1997, 7, 355-361; White, S., Szewczyk, J. W., Turner, J. M., Baird, E. E., and Dervan, P. B., Recognition of the Four Watson-Crick Base Pairs in the DNA Minor Groove by Synthetic Ligands, Nature, 1998, 391, 468-471; Dervan, P. B. and Burli, R. W., Sequence-specific DNA Recognition by Polyamides, Curr. Opin. Chem. Biol., 1999, 3, 688-693. Attachment of the radical cleaving moiety, MPE Fe(II), to the pyrrolopeptide-based agents lead to the design of sequence specific DNA cleaving agents. See Herzberg, R. P. and Dervan, P. B., Cleavage of Double Helical DNA by (Methidiumpropyl-EDTA) Iron (II), J. Am. Chem. Soc., 1982, 104, 313-315 ;Van Dyke, M. W., Herzberg, R. P., and Dervan, P. B., Map of Distamycin, Netropsin, and Actinomycin Binding Sites on Heterogeneous DNA: DNA Cleavage-Inhibition Patterns with Methidiumpropyl-EDTA. Fe (II), Proc. Natl. Acad. Sci. U.S.A., 1982, 79, 5470-5474 ;Taylor, J. S., Schultz, P. G., and Dervan, P. B., DNA Affinity Cleaving Sequence Specific Cleavage of DNA by Distamycin-EDTA. Fe (II), Tetrahedron 1984, 40, 457-465. Sequence-specific DNA cleaving agents are useful for DNA sequence determination as well as chromosome and gene analysis. See Dervan, P. B. and Burli, R. W., Sequence-specific DNA Recognition by Polyamides Curr. Opin. Chem. Biol. 1999, 3, 688-693

The DNA major groove has also been utilized for the design of sequence specific recognition agents. Usually DNA strands or synthetic analogues thereof recognize the major groove by Hoogsteen base pairing to afford a triple helix structure. See Nielsen, P. E. Antisense properties of peptide nucleic acid 2000, 156-164; Nielsen, P. E., DNA Analogues with Nonphosphodiester Backbones, Annu. Rev. Biophys. Biomol. Struct., 1995, 24, 167-183; Majumdar, A., Khorlin, A., Dyatkina, N., Lin, F. L. M., Powell, J., Liu, J., Feiz, Z. Z., Khripine, Y., Watanabe, K. A., George, J., Glazer, P. M., and Seidman, M. M., Targeted Gene Knockout mediated by Triple Helix Forming Oligonucleotides, Nat. Genet., 1998, 20, 212-214; Thurston, D. E., Nucleic Acid Targeting: Therapeutic Strategies for the 21st Century, Brit. J. Cancer., 1999, 80, 65-85; Ren, J. S. and Chaires, J. B., Sequence and Structural Selectivity of Nucleic Acid Binding Ligands, Biochemistry, 1999, 38, 16067-16075; Crooke, S. T. Molecular Mechanisms of Action of Antisense Drugs, Bba Gene. Struct. Express., 1999, 1489, 31-44. Because DNA recognition at the minor and major groove has value in cancer chemotherapy and molecular biology, efforts in this area will no doubt continue into the new millennium. See Thurston, D. E., Nucleic Acid Targeting: Therapeutic Strategies for the 21st Century, Brit. J. Cancer., 1999, 80, 65-85.

This laboratory has been involved in the design of major groove recognition agents that cleave DNA at the phosphate backbone. See Huang, X., Suleman, A., and Skibo, E. B., Rational Design of Pyrrolo[1,2-a ]benzimidazole Based Antitumor Agents Targeting the DNA Major Groove, Bioorg. Chem., 2000, Accepted Dec. Issue; Schulz, W. G., Nieman, R. A., and Skibo, E. B., Evidence for DNA Phosphate Backbone Alkylation and Cleavage by Pyrrolo[1,2a]-albenzimidazoles, Small Molecules Capable of Causing Sequence Specific Phosphodiester Bond Hydrolysis, Proc. Natl. Acad. Sci. U.S.A., 1995, 92, 11854-11858; Skibo, E. B. and Schulz, W. G. Pyrrolo[1,2-a]benzimidazole-Based Aziridinyl Quinones. A New Class of DNA Cleaving Agent Exhibiting G and A Base Specificity, J. Med. Chem., 1993, 36, 3050-3055. Currently, new reactive species capable of recognizing and cleaving at specific sites in the DNA major groove are being developed.

This specification describes the chemistry and DNA alkylating/cleaving properties of the aziridinyl eneimine and of the cyclopent[b]indole methide species shown in Chart 1. Either species has the capacity to trap two nucleophiles resulting in a crosslinking reaction.

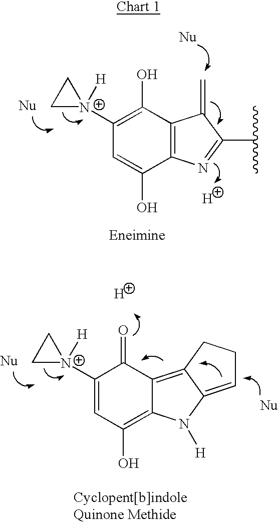

These reactive species were considered to be responsible for the cytotoxic and antitumor properties of indole and cyclopent[b]indole based aziridinyl quinones. See Xing, C., Skibo, E. B., and Dorr, R. T., Aziridinyl Quinone Antitumor Agents based on Indoles and Cyclopent[b]indoles: Structure Activity Relationships for Cytotoxicity and Antitumor Activity, J. Med. Chem., 2001, 44, Submitted; Xing, C. G., Wu, P., Skibo, E. B., and Dorr, R. T., Design of Cancer-Specific Antitumor Agents Based on Aziridlinylcyclopent[b]indoloquinones, J. Med. Chem., 2000, 43, 457-466

### SUMMARY OF THE INVENTION

This invention describes the formation and build-up of reactive species in solution able to react with DNA selectively. Novel aspects of this invention include:

Crosslinking of the G-base by N(7) and 3′-phosphate alkylation. These alkylation products permit the controlled cleavage of DNA and the identification of DNA sequences.

Recognition of 3′-GT-5′ and 3′-GGA-5′ sequences with small molecules.

Rapid determination of hydrolytic DNA cleavage chemistry using the ¹³C-labelling developed in this laboratory.

Eventually, more functionalized versions of these indole and cyclopent[b]indole-based aziridinyl quinones will permit the recognition and cleavage of specific sequences at the major groove of DNA.

The indole (1) and cyclopent[b]indole (2) systems shown in Chart 2 below were previously prepared and evaluated as antitumor agents possessing DNA alkylating capability. See Xing, C. G., Skibo, E. B., and Dorr, R. T.; Aziridinyl Quinone Antitumor Agents Based on Indoles and Cyclopent[b]indoles: Structure Activity Relationships for Cytotoxicity and Antitumor Activity, J. Med. Chem., 2001, 44. The cytotoxicity of both systems prompted the detailed study of their DNA recognition and alkylation properties described herein. Both systems require two-electron reduction to the hydroquinone in order to activate the alkylating centers. Hydroquinone formation activates the aziridinyl group as an alkylating center by permitting nitrogen protonation at neutrality. See Skibo, E. B. and Schulz, W. G., Pyrrolo[1,2-a]benzimidazole-Based Aziridinyl Quinones. A New Class of DNA Cleaving Agent Exhibiting G and A Base Specificity, J. Med. Chem., 1993, 36, 3050-3055; Schulz, W. G., Nieman, R. A., and Skibo, E. B., Evidence for DNA Phosphate Backbone Alkylation and Cleavage by Pyrrolo[1,2-a] benzimidazoles, Small Molecules Capable of Causing Sequence Specific Phosphodiester Bond Hydrolysis, Proc. Natl. Acad. Sci. U.S.A., 1995, 92, 11854-11858; Gutierrez, P. L., The Metabolism of Quinone-Containing Alkylating Agents: Free Radical Production and Measurement, Front. Biosci., 2000, Vol 5, D629-D638. Reduction also activates the acetate as a leaving group resulting in eneimine or quinone methide formation. The quinone methide species is an alkylating agent known to trap nucleophiles on DNA. See Kang, H. M. and Rokita, S. E., Site-Specific and Photo-Induced Alkylation of DNA by a Dimethylanthraquinone-oligodeoxynucleotide Conjugate Nucleic Acids Res., 1996, 24, 3896-3902; Zeng, Q. P. and Rokita, S. E., Tandem Quinone Methide Generation for Cross-linking DNA, J. Org. Chem., 1996, 61, 9080-9081; Rokita, S. E., Yang, J. H., Pande, P., and Greenberg, W. A., Quinone Methide Alkylation of Deoxycytidine, J. Org. Chem., 1997, 62, 3010-3012; Lemus, R. L. and Skibo, E. B., Studies of Extended Quinone Methides. Design of Reductive Alkylating Agents Based on the Quinazoline Ring System, J. Org. Chem., 1988, 53, 6099-6105; Skibo, E. B., Formation and Fate of Benzimidazole-Based Quinone Methides. Influence of pH on Quinone Methide Fate, J. Org. Chem., 1992, 57, 5874-5878; Skibo, E. B., Studies of Extended Quinone Methides, The Hydrolysis Mechanism of 1-Methyl-2-(bromomethyl)-4,7-dihydroxybenzimidazole, J. Org. Chem., 1986, 51, 522-527; Zhou, Q. B. and Turnbull, K. D., Phosphodiester Alkylation with a Quinone Methide, J. Org. Chem., 1999, 64, 2847-2851. The eneimine apecies is related to the mitosene iminium ion known to alkylate DNA nucleophiles. See Franck, R. W. and Tomasz, M., The Chemistry of Mitomycins, 1990, 379-394; Boruah, R. C. and Skibo, E. B., Determination of the pKa Values for the Mitomycin C Redox Couple by Tritration, pH Rate Profile, and Nemst-Clark Fits. Studies of Methanol Elimination, Carbocation Formation, and the Carbocation/Quinone Methide Equilibrium, J. Org. Chem., 1995, 60, 2232-2243; Ouyang, A. and Skibo, E. B., The Iminium Ion Chemistry of Mitosene DNA Alkylating Agents. Enriched ¹³C-NMR Studies, Biochemistry, 2000, 39, 5817-5830. The metabolism of 3-methylindole in fact involves the formation of the eneimine species (also called the methylene imine) that traps nucleophiles. See Skiles, Gary L. and Yost, Garold S., Mechanistic Studies on the Cytochrome P450-Catalyzed Dehydrogenation of 3-Methylindole, Chem. Res. Toxicol., 1996, 9, 291-7; Ruangyuttikam, Werawan, Skiles, Gary L. and Yost, Garold S., Identification of a Cysteinyl Adduct of Oxidized 3-methylindole from Goat Lung and Human Liver Microsomal Proteins, Chem. Res. Toxicol., 1992, 5, 713-19.

Thus 1a is potentially a triple alkylating agent capable of forming a quinone methide, an eneimine, and a protonated aziridine while 1b and 1c each have one or two alkylating center removed. Similarily, 2a and 2b possess either two or one alkylating centers respectively. The parallel study of these compounds will provide insights into how these multiple alkylating agents interact with DNA. The ¹³C-labeled analogues shown in the inset of Chart 2 were also prepared in order to assess the hydrolytic and DNA alkylation chemistry of 1. The placement of ¹³C at reacting centers permits the rapid assessment of the fate of highly reactive species. See Ouyang, A. and Skibo, E. B., The Iminium Ion Chemistry of Mitosene DNA Alkylating Agents. Enriched ¹³C-NMR Studies, Biochemistry, 2000, 39, 5817-5830. The number of enriched ¹³C resonances and their chemical shifts provide insights into the number and type of compounds formed in alkylation reactions.

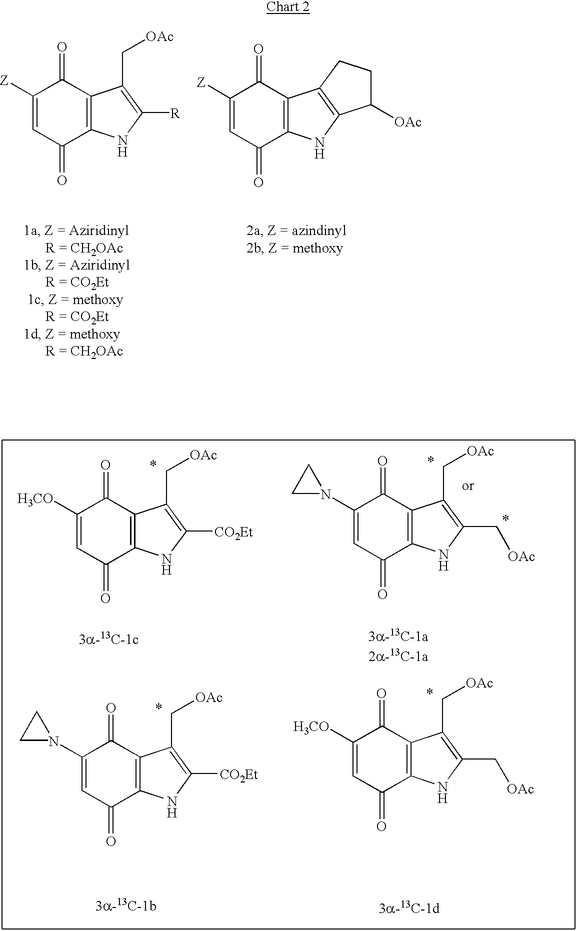

### BRIEF DESCRIPTION OF THE DRAWINGS

FIG. 1. The ¹³C NMR spectrum of eneimine formation from two-electron reduced 3α-¹³C-1c.

FIG. 2. The ¹³C NMR spectrum of dimer 23 from the hydrolysis of 3α-¹³C-1c.

FIG. 3. The ¹³C NMR spectrum of dimer 20 from the hydrolysis of reduced 3α-¹³C-1c.

FIG. 4. The ¹³C NMR spectrum of trimer 21 from the hydrolysis of reduced 3α-¹³C-1c.

FIG. 5. The ¹³C NMR spectrum of alkylated hexamer 5′-ATGCAT-3′ obtained by treatment of the DNA with two-electron reduced 3α-¹³C-1c.

FIG. 6. ¹³C NMR Spectra of native hexamer 5′-ATGCAT-3′ (part A) and alkylation products obtained by treatment of the hexamer with two-electron reduced 2α-¹³C-1a (part B) and 3α-¹³C-1a (part C).

FIG. 7. The PAGE gel shown in FIG. 7 reveals that the reductive cleavage of DNA by 1a occurred at both the guanine (N-7) and the 3′ guanosine phosphate.

FIG. 8. The PAGE gel in FIG. 8 compares the reductive cleavage by 1a with cleavage by 1b,d and an analogue without a 3α leaving group. Lanes 1 and 2 show piperidine and hydrolytic cleavage respectively by reduced 1a.

FIG. 9. Autoradiogram of the reductive cleavage of a 5′-³²p end-labeled 514 bps restriction fragment from pBR322 DNA (Ecor I/ Rsa I) by 2a,b and the reported 6-aziridinyl isomer of 2a.

### DETAILED DESCRIPTION OF THE INVENTION

Synthesis

Incorporation of the ¹³C -label at the 2α-position to afford 2¹³C-1a involved the procedure outlined in Scheme 1. Procedures for the conversion of 5-methoxyindole to 6 are provided in the Experimental Section. The conversion of 6 to 7 and the eventual preparation of 2α-¹³C-1a were carried out employing procedures previously reported for the unlabeled analogues. The incorporation of the ¹³C-label at the 3α-position was carried out by Vilsmeier formylation of 8 with ¹³C-DMF to afford 9. Multistep conversion of 9 to 3α-¹³C-1a-d was carried out employing reported procedures. See Xing, C., Skibo, E. B., and Dorr, R. T., Aziridinyl Quinone Antitumor Agents Based on Indoles and Cyclopent[b]indoles: Structure Activity Relationships for Cytotoxicity and Antitumor Activity, J. Med. Chem., 2001, 44.

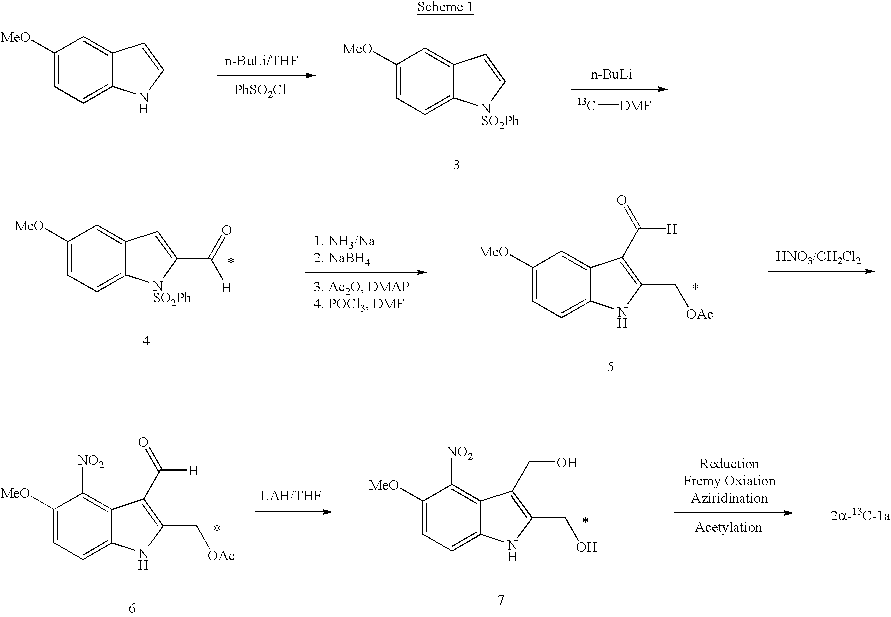

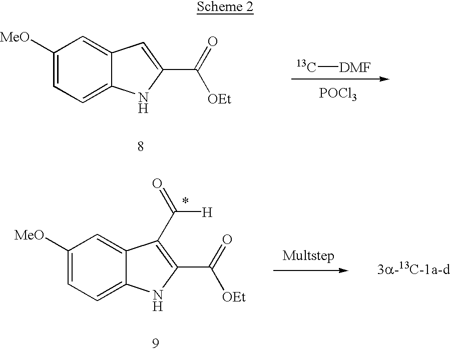

Eneimine Formation and Fate

The 3α-¹³C labeled derivatives of 1c and 1d were used to verify that the eneimine can build-up in aqueous media and undergo complex reactions. Both of these compounds do not possess the aziridinyl group, so that only eneimine formation and fate can be studied.

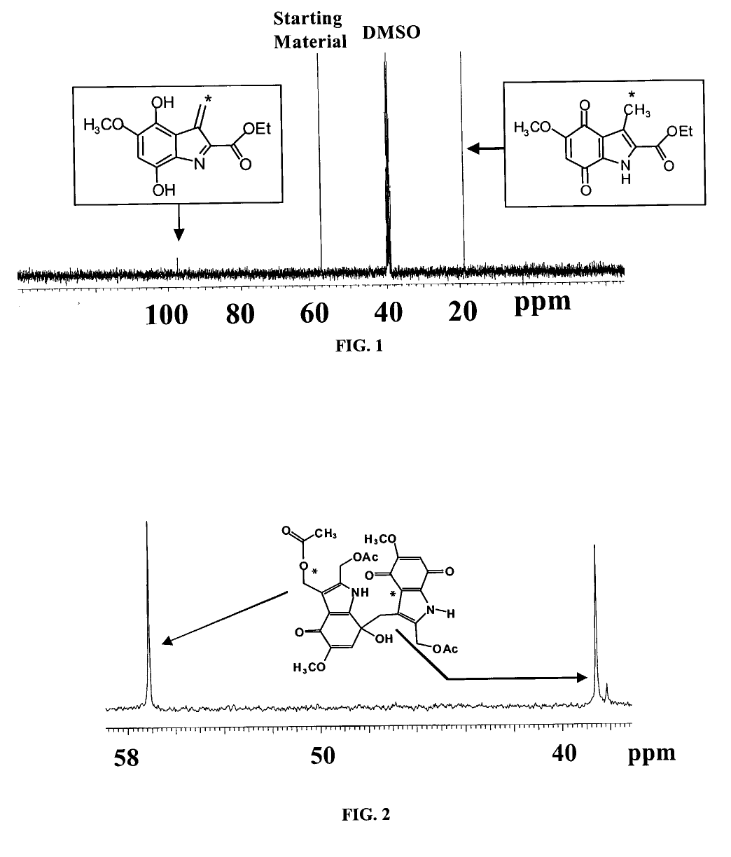

Shown in FIG. 1 is the 100 MHz ¹³C-NMR spectrum (obtained on a 400 MHz instrument) of 2-electron reduced 3α-¹³C-1c obtained after 4 h of acquisitions. The presence of the methylene center of the eneimine is apparent at 98 ppm, along with starting material at 58 ppm and a reaction product at 18 ppm. The reaction product was isolated from a preparative reaction described below.

FIG. 1

The ¹³C NMR spectrum of eneimine formation from two-electron reduced 3α-¹³C-1c. The reaction contained 2 mg of starting material, 0.5 mL of d₆-DMSO, and 0.5 mL pD=7.2 0.1M phosphate D₂O buffer.

Reduction of 3α-¹³C-1c and incubation in methanol afforded the complex mixture of products whose structures are shown in Scheme 3. An internal redox reaction (transfer of H₂ from the hydroquinone to the eneimine) results in formation of 10 and its transesterification product 11. Product 10 is the same as that observed in the NMR tube reaction described above. The eneimime also traps methanol solvent to afford 12 as its hydroquinone, which then affords the corresponding quinone upon aerobic workup. However, most of the eneimine traps hydroquinone nucleophiles to form dimers 13-15 through bond formation at C-5 or C-7.

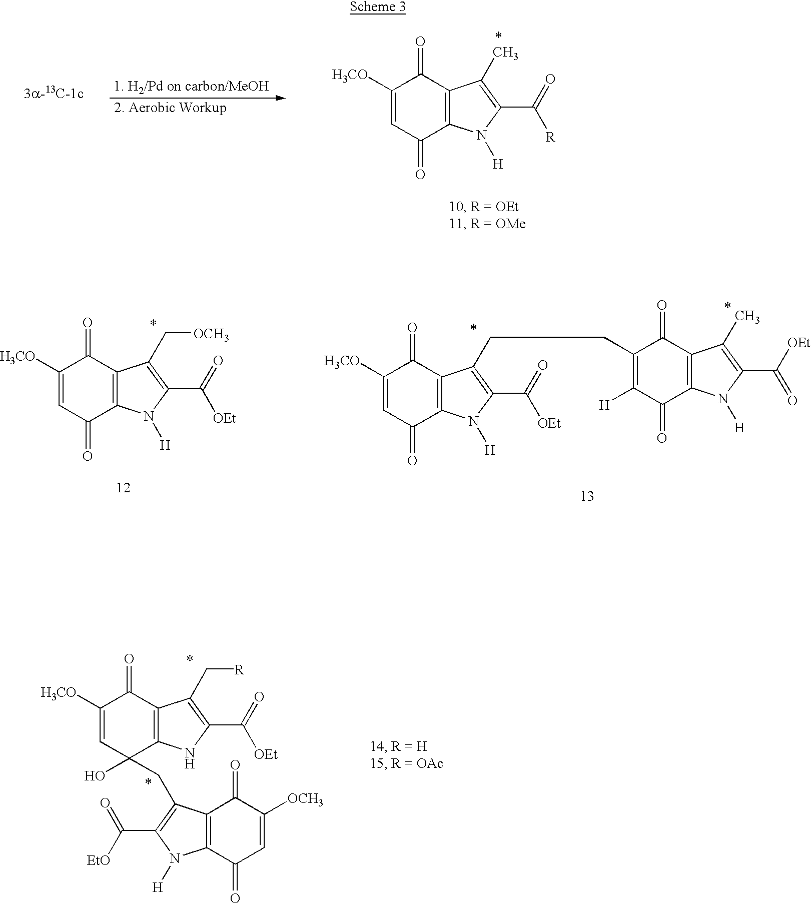

Reduction of 3α-¹³C-1d and incubation in methanol likewise afforded the complex mixture of products whose structures are shown in Scheme 4. The products arising from the internal redox reaction, 16 and 17, are minor components of the mixture. The hydroquinone is the major nucleophile in solution resulting in the formation of dimers (19-20 and 22-24) and trimer 21 in addition to methanol trapping product 18.

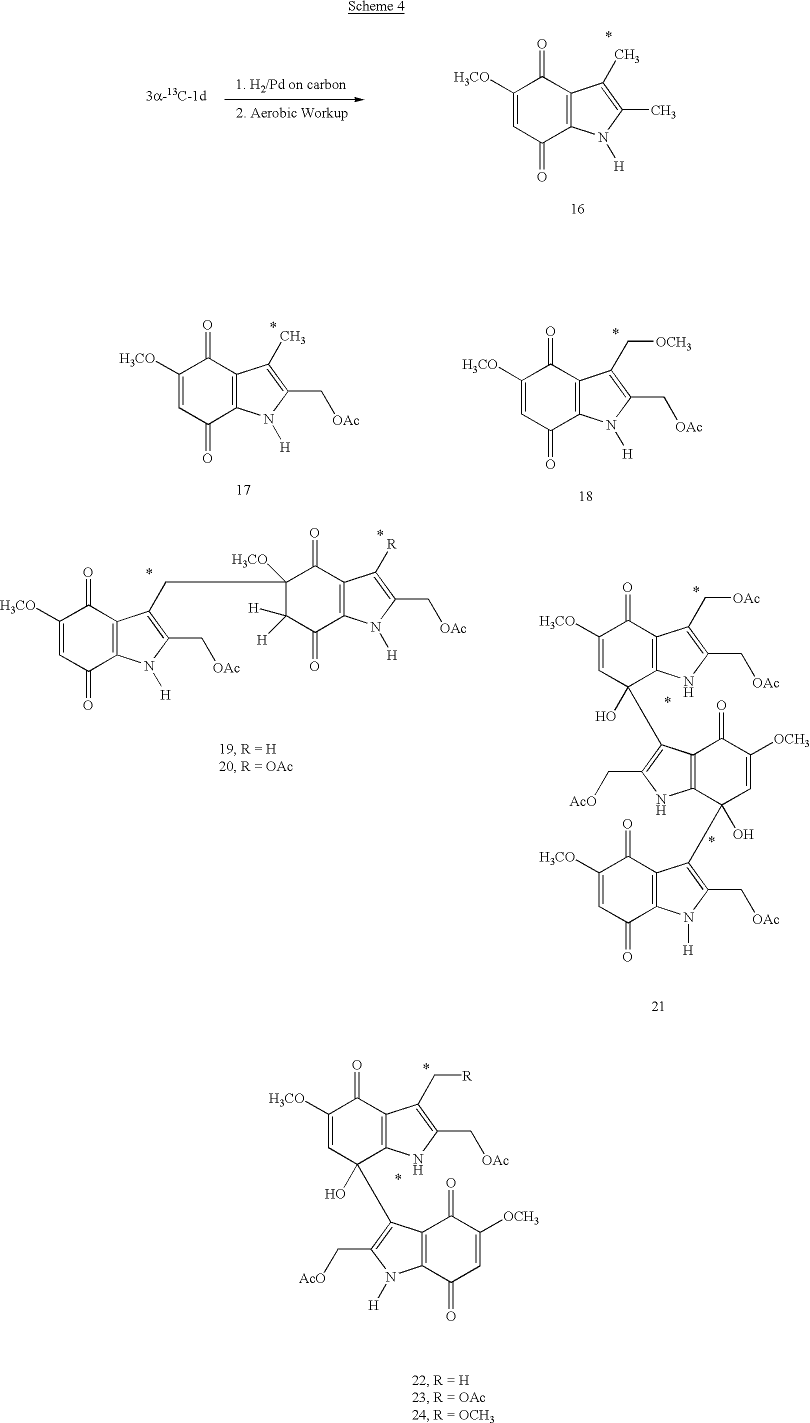

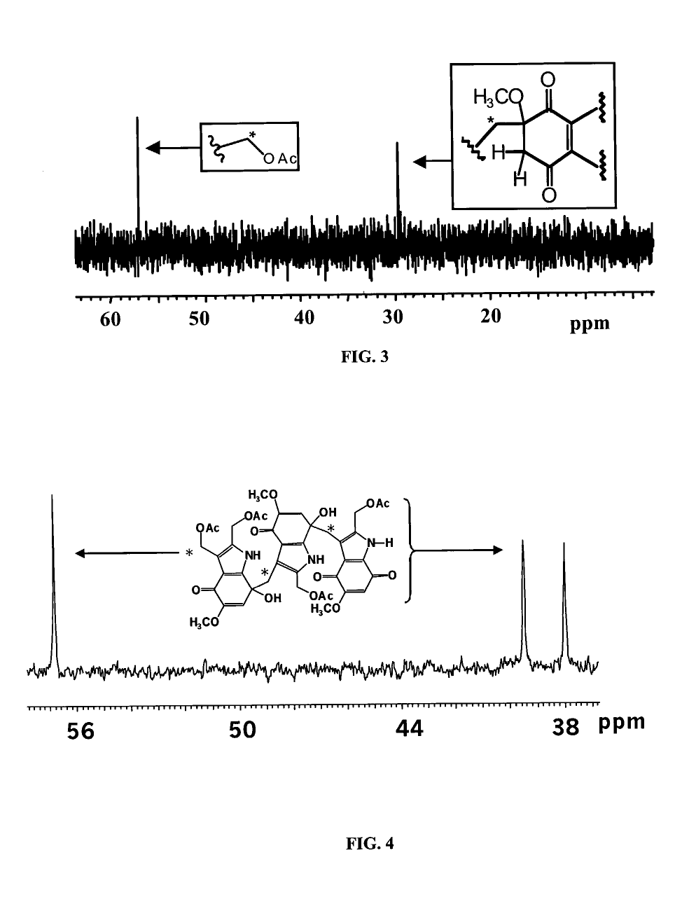

The structure of the dimers and trimer 21 in Schemes 3 and 4 were derived from ¹HNMR, ¹³CNMR, COSY, HMQC, and HMBC. Furthermore, the structure of trimer 21 was confirmed by X-ray crystallography. The incorporation of ¹³C into the 3α position proved valuable in structural determinations, as illustrated by FIGS. 2-4. The ¹³C-NMR of dimers 23 and 20 shown in FIGS. 2 and 3 reveal the presence of the acetoxymethyl (57 ppm) as well as the linking methylene (38 ppm). The trimeric structure of 21 was readily assessed from the ¹³C-NMR shown in FIG. 4 and shows two linking methylenes as well as the terminal acetoxymethyl. Methylene links resulting from direct carbonyl addition possess ¹³C chemical shifts in the 35-38 ppm range. Previous studies of polymeric species derived from iminium ions showed that methylene links resulting from a Michael addition, such as those in 19 and 20, possess ¹³C chemical shifts in the 25-30 ppm range. See Ouyang, A. and Skibo, E. B., The Iminium Ion Chemistry of Mitosene DNA Alkylating Agents. Enriched ¹³C-NMR Studies, Biochemistry, 2000, 39, 5817-5830.

FIG. 2

The ¹³C NMR spectrum of dimer 23 from the hydrolysis of 3α-¹³C-1c.

FIG. 3

The ¹³C NMR spectrum of dimer 20 from the hydrolysis of reduced 3α-¹³C-1c.

FIG. 4

The ¹³C NMR spectrum of trimer 21 from the hydrolysis of reduced 3α-¹³-C-1c.

The dimer and trimer structures shown in Schemes 3 and 4 can be readily determined spectroscopically, in spite of their complexity, as summarized below as follows.

Either the direct carbonyl or the Michael addition processes afford a stereocenter adjacent to the ¹³C-methylene center. As a result, the H-NMR spectrum of this methylene center shows two doublet of doublets. The methylene protons are chemically nonequivalent due to the adjacent stereocenter resulting in gem splitting (doublet of doublets). The ¹³C carbon splits the doublets of doublets into two sets with a J value typically of 130 Hz. The ¹³C-enriched HMQC spectrum of the dimer or trimer product readily identifies the H-NMR chemical shifts of the ¹³C-methylene center. Inspection of the COSY spectrum will show two adjacent “boxes” corresponding to the doublets of doublets. A COSY spectrum of a mitosene dimer showing this feature was published recently. See Ouyang, A. and Skibo, E. B., The Iminium Ion Chemistry of Mitosene DNA Alkylating Agents. Enriched ¹³C-NMR Studies., Biochemistry, 2000, 39, 5817-5830.

The ¹³C-enriched HMBC spectrum shows two-bond coupling between the ¹³C-methylene center and a methylene center adjacent to a carbonyl indicating a Michael addition product.

The indole NH protons are readily apparent between 9-10 ppm in the H-NMR spectrum indicating that alkylation of this center had not occurred as well as the number of indole units (two for dimer and three for trimer). Each indole monomer possesses a 100% enriched ¹³C center, and merely counting the ¹³C centers provides the number of repeating indole units.

The chemistry of the eneimine and the parent indole system can be readily explained by referring to the electron density of the indole ring based on molecular orbital calculations, inset of Scheme 5. These chemical explanations are outlined below in conjunction with Schemes 5 and 6.

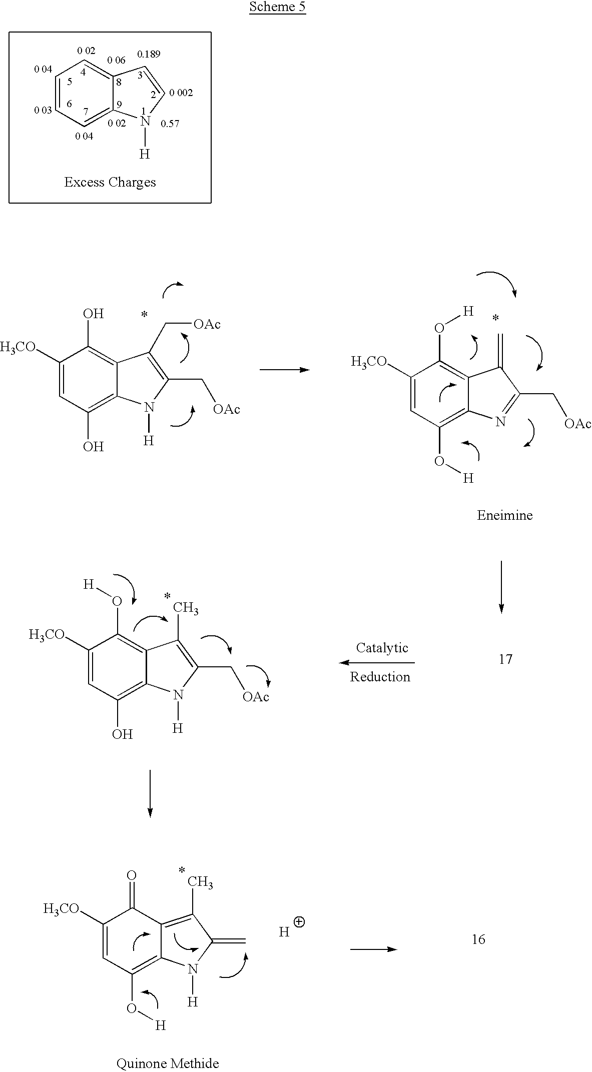

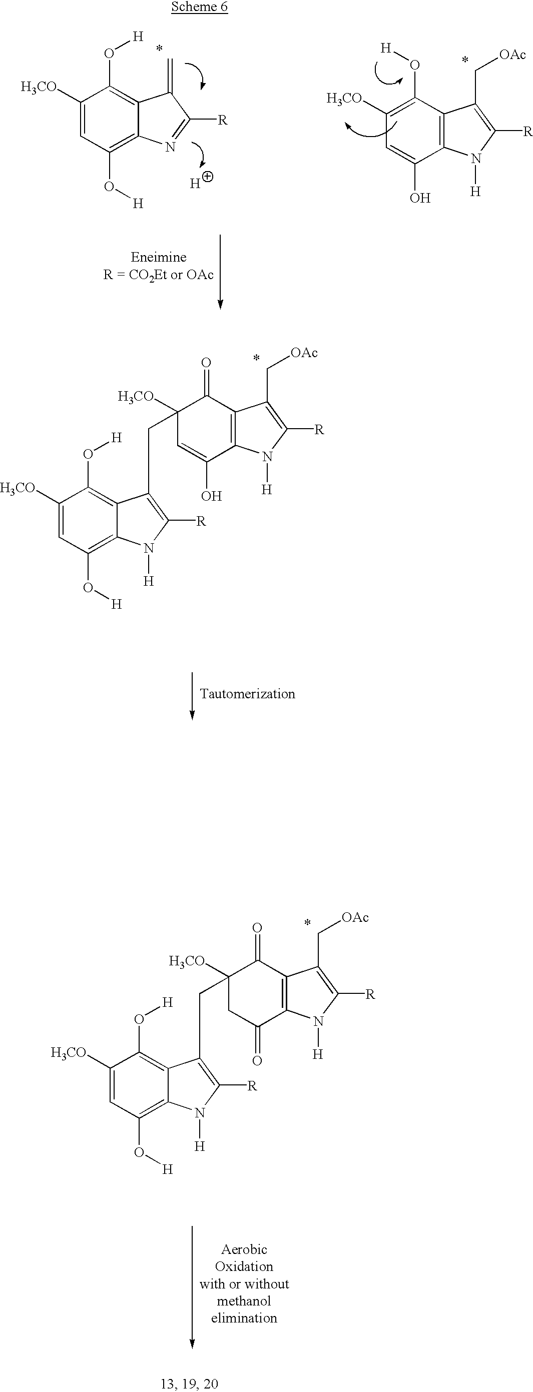

Electron density for the indole system is higher at the 3-position than at the 2-position resulting in almost exclusive eneimine formation. Thus, the formation of the quinone methide species by acetate elimination from the 2-position contributes only if eneimine formation is not possible.

Excess electron density resides on positions 5 and 7 of the indole ring results in electrophilic attack by the eneimine at these positions. The mechanism for the formation of the 5-linked dimers 13, 19, 20 is illustrated in Scheme 6. Trapping of the eneimine electrophile by the 3- and 8-positions was not observed in this study. A previous study reported trapping of the iminium ion by the 3-position followed by loss of formaldehyde; See Ouyang, A. and Skibo, E. B., The Iminium Ion Chemistry of Mitosene DNA Alkylating Agents. Enriched ¹³C-NMR Studies, Biochemistry, 2000, 39, 5817-5830. Trapping by the 8-position has never been observed and it is believed that this is probably because the product can never aromatize. The high specificity of the eneimine for nucleophilic sites on the indole system suggests that this electrophilic species will selectively trap nucleophiles on DNA.

Cyclopent[b]indole Quinone Methide

The formation of this reactive species has not been documented as well as the eneimine has, because ¹³C incorporation at the electrophilic center has yet to be accomplished. Reduction of 2a in anaerobic buffer produced the dimeric species 25a,b suggesting that the quinone methide species can form in solution (see mechanism in the inset of Scheme 7). Modeling studies, however, suggested that the quinone methide will be stable. See Xing, C. G., Wu, P., Skibo, E. B., and Dorr, R. T., Design of Cancer-Specific Antitumor Agents Based on Aziridlinylcyclopent[b]indoloquinones, J. Med. Chem., 2000, 43, 457-466. The cyclopent[b]indole quinone methide proved to be a highly selective alkylating and cleaving agent of DNA.

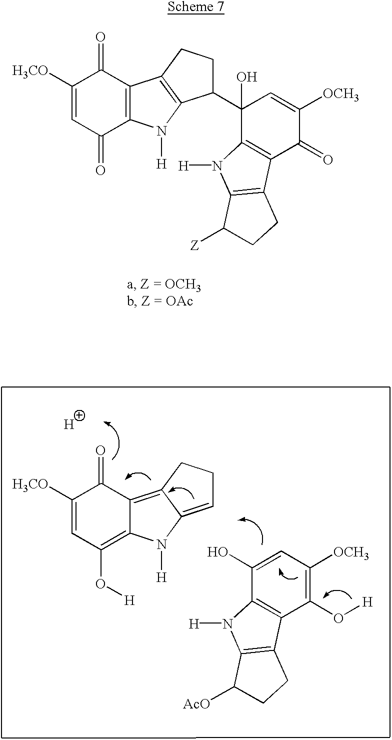

Eneimine Reaction with DNA

The most reactive nucleophilic center of DNA is the guanine N(7) position. See Kim, H. S. and LeBreton, P., UV Photoelectron and ab initio Quantum Mechanical Characterization of Valence Electrons in Na⁺-water-2′-Deoxyguanosine 5′-Phosphate Clusters: Electronic Influences on DNA Alkylation by Methylating and Ethylating Carcinogens, Proc. Natl. Acad. Sci. USA., 1994, 91, 3725-3729; Kim, H. S., Yu, M., Jaing, Q., and LeBreton, P. R., UV Photoelectron and Ab Initio Quantum Mechanical Characterization of 2′-Deoxyguanosine 5′-Phosphate: Electronic Influences on DNA Alkylation Patterns, J. Am. Chem. Soc., 1993, 115, 6169-6183; Broch, H., Hamza, A., and Vasilescu, D., Quantum Molecular Modeling of the Interaction Between Quanine and Alkylating Agents-I-Sulfur Mustard, J. Biomol. Struct. Dyn., 1996, 13, 903-914; Hamza, A., Broch, H., and Vasilescu, D. Quantum Molecular Modeling of the Interaction Between Quanine and Alkylating Agents-2-Nitrogen Mustard, J. Biomol. Struct. Dyn., 1996, 13, 915-924.

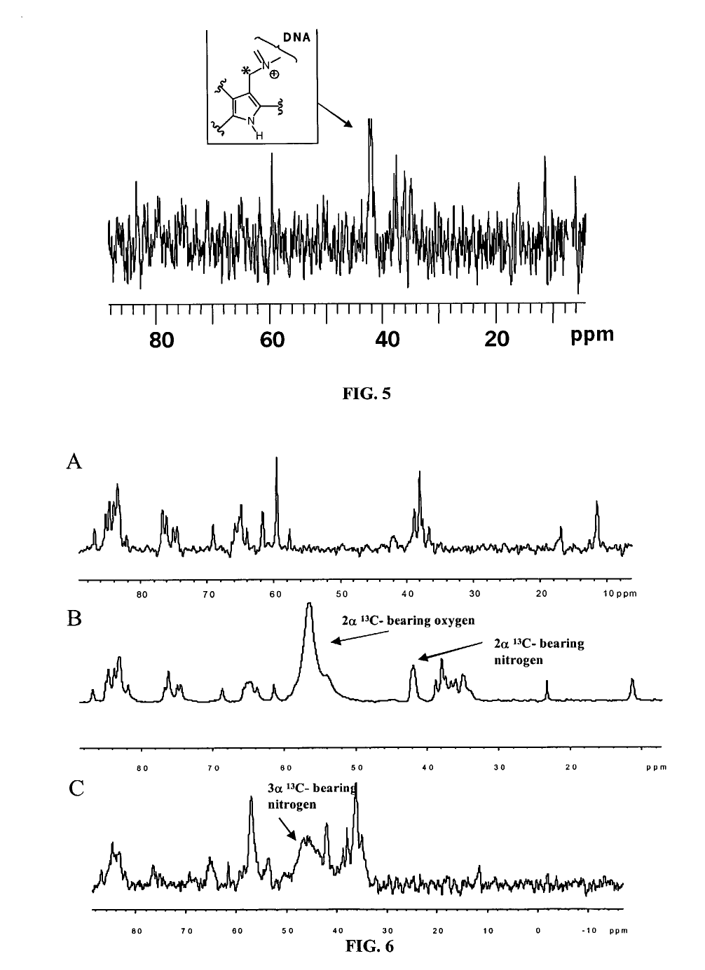

The eneimine electrophile should selectively alkylate DNA at the guanine N(7) position. In order to determine the DNA alkylation site, hexamer DNA with the sequence ATGCAT was treated with two-electron reduced 3α-¹³C-1b. The DNA hexamer was first purified by 20% PAGE as described in the Experimental Section and then reductively alkylated. The reduction was carried out catalytically and the DNA was recovered from precipitation. The ¹³C NMR spectrum of this DNA was compared with that of the native hexamer. The alkylated DNA spectrum shown in FIG. 5 has a strong peak at 42 ppm, indicating that the ¹³C-methylene is bound to a nitrogen. This result is consistent with alkylation of adenine or guanine N(7) as well the 2-amino of guanine. PAGE studies described herein indicated that the N(7) positions are the alkylation sites.

FIG. 5

The ¹³C NMR spectrum of alkylated hexamer 5′-ATGCAT-3′ obtained by treatment of the DNA with two-electron reduced 3α-¹³C-1c.

The hexamer was also treated with reduced 3α-¹³C-1a and 2α-¹³C-1a and ¹³C-NMR spectra of the alkylation products were obtained and compared with the ¹³C natural abundance spectrum of the hexamer, FIG. 6. The series of spectra in this figure illustrate the limitation of using enriched ¹³C NMR to study DNA alkylation reactions. The formation of multiple alkylation products as well as low yields could dilute the incorporated ¹³C labels to the point that they are as intense as the natural abundance ¹³C spectrum. The solution is to compare the natural abundance and enriched spectra and note differences. Comparison of the natural abundance ¹³C NMR spectrum (part A) with the ¹³C spectrum of 2α-labeled alkylating agent 2α-¹³C-1a (part B) reveals that the oxygen of acetate was still attached at the 2α-¹³C center. In contrast, the 3α-labeled alkylating agent 3α-¹³C-1a afforded a ¹³C spectrum showing that nitrogen alkylation had occurred at this position. These results are consistent with elimination of acetate from the 3α-position rather than from the 2α-position as observed in the hydrolysis study shown in Scheme 6.

FIG. 6

¹³C NMR Spectra of native hexamer 5′-ATGCAT-3′ (part A) and alkylation products obtained by treatment of the hexamer with two-electron reduced 2α-¹³C-1a (part B) and 3α-³C-1a (part C).

The PAGE gel shown in FIG. 7 reveals that the reductive cleavage of DNA by 1a occurred at both the guanine (N-7) and the 3′ guanosine phosphate. The alkylation of either the adenine or guanine N(7) was determined by piperidine treatment according to Maxam and Gilbert. See Maxam, A. M. and Gilbert, W., Sequencing End-Labeled DNA with Base-Specific Chemical Cleavages, Methods Enzymol., 1980, 65, 499-560. The piperidine treatment also hydrolyzed the phosphotriester resulting from phosphate alkylation without detectable backbone cleavage. In contrast, the treatment of the alkylated DNA with basic loading buffer resulted in only phosphate backbone cleavage without detectable cleavage via N(7) adducts. See Schulz, W. G., Nieman, R. A., and Skibo, E. B., Evidence for DNA Phosphate Backbone Alkylation and Cleavage by Pyrrolo[1,2-a] benzimidazoles, Small Molecules Capable of Causing Sequence Specific Phosphodiester Bond Hydrolysis, Proc. Natl. Acad. Sci. U.S.A., 1995, 92, 11854-11858.

FIG. 7 Legend

Autoradiogram of the 1α-mediated reductive cleavage of a 5′-³²p end-labeled 514 bps restriction fragment from pBR322 DNA (Ecor I/ Rsa I)

| | Compound | Conditions | Cleavage Pattern |
|---|---|---|---|
| Lane 1 | 1a | Catalytic reductive | G |
| | | activation, piperidine | |
| | | cleavage | |
| Lane 2 | 1a | Catalytic reductive | G + 1 (3′ direction) |
| | | activation, hydrolytic | |
| | | cleavage | |
| Lane 3 | 1a | NaBH₄ reductive activation, | G & A |
| | | piperidine cleavage | |
| Lane 4 | 1a | NaBH₄ reductive activation, | G + 1 (3′ direction) |
| | | hydrolytic cleavage | A + 1 (3′ direction) |
| Lane 5 | 1a | NaBH₄ reductive activation, | G & A |
| | | piperidine cleavage | |
| Lane 6 | 1a | NaBH₄ reductive activation, | G + 1 (3′ direction) |
| | | hydrolytic cleavage | A + 1 (3′ direction) |
| Lane 7 | DMS | Piperdine cleavage | G & A |

The first two lanes of FIG. 7 show the results of a limited alkylation reaction followed by piperidine and hydrolytic cleavage respectively. The ladder in the first lane shows the presence of specific guanine N(7) alkylation while the ladder in the second lane shows phosphate alkylation in the G+1 direction (of 5′-labeled DNA). The PAGE results and the ¹³C-NMR spectrum shown in FIG. 5 support a process where the eneimine intermediate selectively reacts with guanine N(7) and the aziridinyl center is then directed to the 3′-guanosine phosphate as shown in Scheme 8.

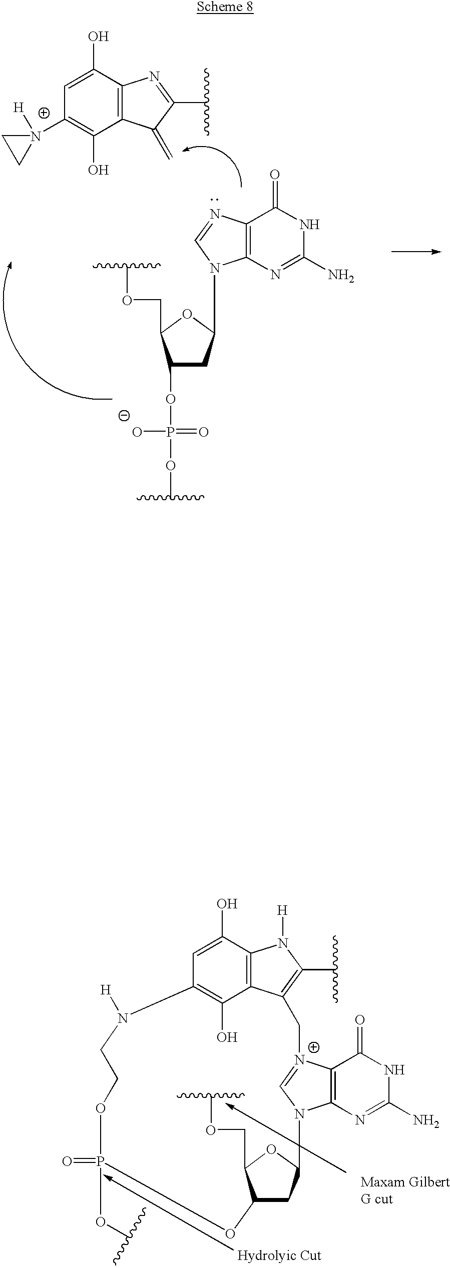

When borohydride reduction was employed, both adenine and guanine N(7) alkylation occurred (lanes 3 and 5) along with alkylation of the corresponding 3′-phophates (G+1 and A+1 cleavage ladders). Reductive activation with borohydride results in the rapid formation of the eneimine alkylating agent. In contrast, the slower catalytic reduction affords limiting amounts of eneimine and hence limited cleavage (lanes 1 and 2).

The PAGE gel in FIG. 8 compares the reductive cleavage by 1a with cleavage by 1b,d and an analogue without a 3α leaving group. Lanes 1 and 2 show piperidine and hydrolytic cleavage respectively by reduced la. These lanes were obtained for comparative purposes and possess the same cleavage patterns as those in FIG. 7.

FIG. 8 Legend

Autoradiogram of the reductive cleavage of a 5′-³²p end-labeled 514 bps restriction fragment from pBR322 DNA (Ecor I/Rsa I) by 1a,b,d and the reported 3-methyl analogue.

| | | |
|---|---|---|
| Compound | Conditions | Cleavage Pattern |

| | | | |
|---|---|---|---|
| Lane 1 | 1a | NaBH₄ reductive activation, | G & A |
| | | piperidine cleavage | |
| Lane 2 | 1a | NaBH₄ reductive activation, | G + 1 (3′ direction) |
| | | hydrolytic cleavage | A + 1 (3′ direction) |
| Lane 3 | 1b | NaBH₄ reductive activation, | G & A |
| | | piperidine cleavage | Preference for A |
| Lane 4 | 1b | NaBH₄ reductive activation, | Some G & A |
| | | hydrolytic cleavage | |
| Lane 5 | 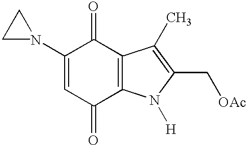 | NaBH₄ reductive activation, piperidine cleavage | Some G & A |
| Lane 6 | 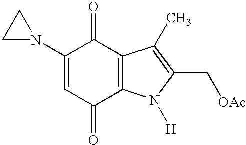 | NaBH₄ reductive activation, hydrolytic cleavage | Weak Cleavage |
| Lane 7 | 1d | NaBH₄ reductive activation, | GT (5′ |
| | | piperidine cleavage | direction) |
| Lane 8 | 1d | NaBH₄ reductive activation, | 3′-GGA-5′ at the |
| | | hydrolytic cleavage | center G |
| Lane 9 | DMS | Piperdine cleavage | G & A |

The change from the 2-acetoxymethyl (1a) to the 2-ethoxycarbonyl group (1b) does not influence eneimine trapping of the adenine and the guanine N(7) nucleophiles (compare Lanes 1 and 3). However, the hydrolysis ladder in lane 4 does not show the G+1 and the A+1 ladder observed for 1a, Lane 2. In some sequences, the piperidine and hydrolysis cleavage patterns of Lane 4 are identical while in other sequences there is no corresponding hydrolytic cleavage. These results suggest that attachment of the 1b eneimine to the N(7) center permits access of the aziridinyl alkylating center to the 5′ phosphate or to neither phosphate. Note that DNA cleavage by N(7) alkylation and piperidine treatment affords the same products as 5′ phosphate hydrolysis.

The piperidine and hydrolysis cleavage ladders shown in Lanes 5 and 6, respectively, were determined for an aziridinyl indoloquinone without a 3α leaving group. The weak hydrolysis ladder in Lane 5 in fact indicates that phosphate alkylation is not an important process when the eneimine species cannot form. These results are consistent with the mechanism whereby the eneimine is the initial DNA alkylating species followed by aziridinyl alkylation of the phosphate backbone.

The cleavage ladders shown in Lanes 7 and 8 show the unusual multibase recognition capability of 1d. The hydrolysis ladder in Lane 8 shows that recognition occurs only at 3′-GGA-5′ with phosphate alkylation and cleavage occurring largely at the center G base. The reason for multibase recognition by such a small molecule may be the presence of dimers and trimers. Recall that the hydrolysis of reduced 1d afforded a variety of such species in good yields, 19-24 in Scheme 4. Any of these species could form a quinone methide or eneimine species capable of alkylating the phosphate backbone or N(7) centers. Investigations are ongoing to determine whether trimer 21 and dimmers could actually recognize and cleave DNA.

Cyclopent[b]indole Quinone Methide Reaction with DNA

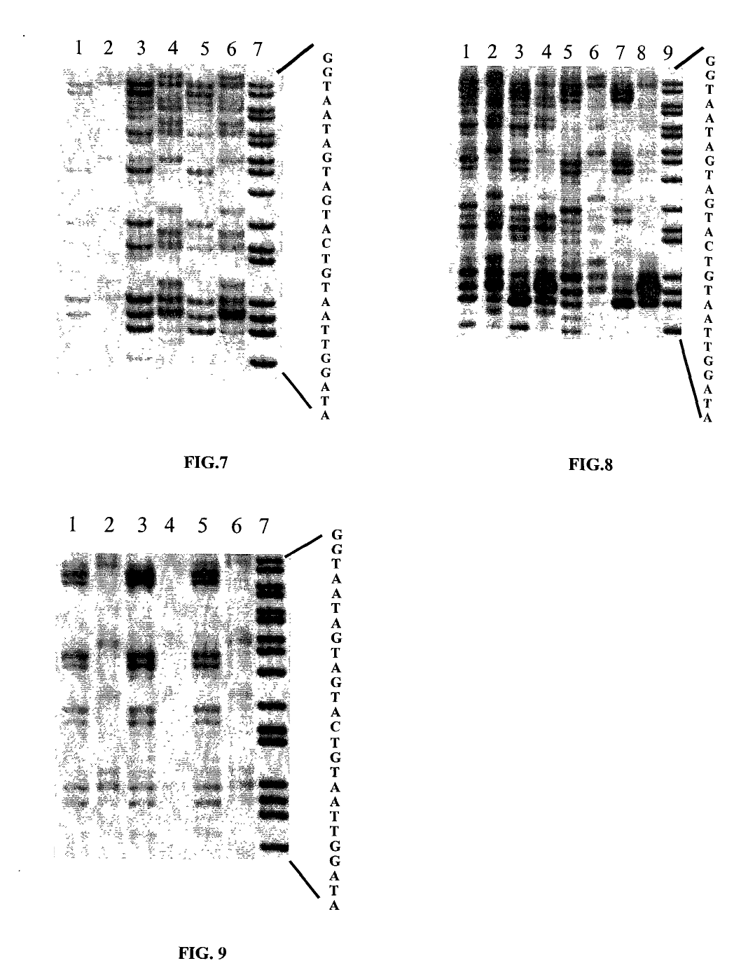

The DNA cleavage ladders obtained with reduced cyclopent[b]indoles 2a,b and an isomer of 2a are shown in FIG. 9. These ladders clearly show that the aziridinyl group is not involved in the alkylation and cleavage process. Thus repositioning of the aziridinyl ring, and even its removal, resulted in the same cleavage pattern. Furthermore, the weak or nonexistent hydrolytic ladders in FIG. 9 reveal the absence of phosphate alkylation. The reduced cyclopent[b]indole recognizes 3′-GT-5′ and cuts at both the G and T bases upon treatment with piperidine.

FIG. 9

Autoradiogram of the reductive cleavage of a 5′-³²p end-labeled 514 bps restriction fragment from pBR322 DNA (Ecor I/ Rsa I) by 2a,b and the reported 6-aziridinyl isomer of 2a.

| | | | Cleavage |
|---|---|---|---|
| | Compound | Conditions | Pattern |
| Lane 1 | 2a | NaBH₄ reductive activation, | G & T of 3′-GT- |
| | | piperidine cleavage | 5′ |
| Lane 2 | 2a | NaBH₄ reductive activation, | Weak Cleavage at |
| | | hydrolytic cleavage | G |
| Lane 3 | 2b | NaBH₄ reductive activation, | G & T of 3′-GT- |
| | | piperidine cleavage | 5′ |
| | | NaBH₄ reductive activation, | No Significant |
| Lane 4 | 2b | hydrolytic cleavage | Cleavage |
| Lane 5 | 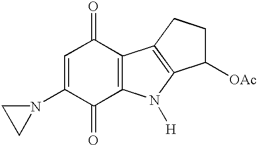 | NaBH₄ reductive activation, piperidine cleavage | G & T of 3′-GT- 5′ |
| Lane 6 | 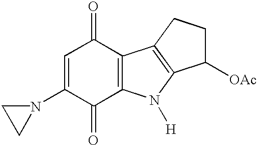 | NaBH₄ reductive activation, hydrolytic cleavage | No Significant Cleavage |
| Lane 7 | DMS | Piperidine cleavage | G & A |

The mechanism of cyclopent[b]indole DNA recognition and cleavage is as not yet completely understood, but insights into these processes were derived at from the PAGE results shown in FIG. 9 and molecular modeling. A cyclopent[b]indole quinone methide would have to recognize the 3′-GT-5′ sequence by interacting with the GC and AT base pairs in the major groove so as to place the alkylating center near the guanine N(7) and the adjacent to the oxygen near the thymine 6-carbonyl. Alkylation of either of these centers would result in hydrolytic removal of either base followed by piperidine-mediated strand cleavage. When the methide center was docked near the guanine N(7) in the 3′-GT-5′ sequence and the resulting complex minimized, the methide was intercalated between the base pairs without any distinctive hydrogen bonding interactions. In addition, the model indicated that placement of the methide near the guanine N(7) will permit reaction with the thymine oxygen.

### Conclusions

DNA recognition agents based on the aziridinyl eneimine and the cyclopent[b]indole methide species have been designed and evaluated utilizing ¹³C NMR and PAGE. The recognition process involved either selective alkylation or intercalating interactions in the major groove. DNA cleavage resulted from phosphate backbone alkylation (hydrolytic cleavage) and N(7)-alkylation (piperidine cleavage), thereby supporting the following conclusions.

The aziridinyl eneimine, resulting from elimination of acetate from the indole 3α position, specifically alkylates the N(7) position of DNA resulting in direction of the aziridinyl alkylating center to either the 3′- or 5′-phosphate of the alkylated base. One reported aziridinyl eneimine was found to be specific for guanine N(7) and this base's 3′-phosphate.

The nonaziridinated indole eneimine was studied in order to gain insights into eneimine chemistry in the absence of other reacting centers. Spectroscopic evidence was obtained for the buildup of the eneimine in solution. Both dimers and trimers are formed by electrophilic attack of the eneimine at either the 5- or 7-positions of the indole ring. NMR and x-ray crystallography were employed to characterize these novel products. The eneimine trimers and dimers appear to be capable of multibase recognition of DNA.

Generation of an indole-base quinone methide by acetate elimination from the 2α-position did not occur either in hydrolysis reactions or in DNA alkylation reactions. Since the indole 3-position is more electron rich than the 2-position, eneimine formation rather than quinone methide formation is the predominate reaction. This conclusion is relevant to the mechanism of indole -based antitumor agents that rely on leaving group elimination from the 2-position, such as WV-15. See Maliepaard, M., deMol, N. J., Tomasz, M., Gargiulo, D., Janssen, L. H. M., vanDuynhoven, J. P. M., vanVelzen, E. J. J., Verboom, W., and Reinhoudt, D. N., Mitosene-DNA Adducts. Characterization of Two Major DNA Monoadducts formed by 1,10-bis(acetoxy)-7-Methoxymitosene Upon Reductive Activation, Biochemistry, 1997, 36, 9211-9220) and E09. See Maliepaard, M., Wolfs, A., Groot, S. E., de Mol, N. J., and Janssen, L. H. M., Indoloquinone E09: DNA Interstrand Cross-linking Upon Reduction by DT-Diaphorase or Xanthine Oxidase, Br. J. Cancer, 1995, 71, 836-839 ;Workman, P., Binger, M., and Kooistra, K. L. Pharmacokinetics, Distribution, and Metabolism of the Novel Bioreductive Alkylating Indoloquinone E09 in Rodents, International Journal of Radiation Oncology Biology Physics, 1992, 22, 713-716).

The cyclopent[b]indole quinone methide forms in solution and can trap itself (dimerize) and trap DNA nucleophiles (gunaine N(7) and the thymine 6-carbonyl oxygen). The aziridinyl group is not required for alkylation and cleavage of DNA; these processes are mediated entirely by the quinone methide species.

The findings enumerated above are starting points for further studies that will lead to new classes of DNA cleaving and recognition agents.

### Experimental Section

The ¹³C NMR spectra was taken on Varian Inova 400 either in Nolorac dual PFG for hexamer or Varian indirect PFG for hydrolytic products at 100 MHz.

Acetylation Procedure for 1 and 2: The reported compounds in Chart 2 (1a, 1b, and 2a) were prepared as previously described Compounds 1c, 1d, and 2b were prepared by acetylation of the previously reported alcohol derivatives. See Xing, C., Skibo, E. B., and Dorr, R. T., Aziridinyl Quinone Antitumor Agents Based on Indoles and Cyclopent[b]indoles: Structure Activity Relationships for Cytotoxicity and Antitumor Activity, J. Med. Chem., 2001, 44, 3545-3562.

To a solution of 0.2 mmol of the alcohol in 5 mL of CH₂Cl₂ and 1 mL of acetone containing 50 mg of DMAP was added 100λ acetic anhydride. The solution was stirred at room temperature for 5 minutes and directly flash chromatographed using ethyl acetate as the eluent. The eluted solution containing the product was washed with NaHCO₃, dried over Na₂SO₄, and vacuum dried. The product was recrystallized from ethyl acetate and hexane to afford a near quantitative yield of the acetylated product. Physical properties of these acetylated products are provided below for 1c, 1d, and 2b:

Ethyl 3-acetoxymethyl-5-methoxyindol-4,7-dione-2-carboxylate (1c): Mp 194-196° C.

TLC(dichloromethane: MeOH 98:2), R_{ƒ}=0.55; ¹HNMR(CDCl₃) δ9.86(1H, bs, indole proton), 5.79(1H, s, 6-proton), 5.84 and 5.28(2H, d, J=168.3 Hz, 3-methylene protons), 4.41(2H, q, J=7.2 Hz, 2-methylene of ethyl), 3.87 (3H, s, 5-methoxy), 2.06(3H, s, 3-acetoxylmethyl), 1.39(3H, t, J=7.2 Hz, 2-methyl of ethyl); ¹³C NMR(CDCl₃) δ55.48 ppm; IR(KBr pellet) 3491, 3354, 3302, 3211, 3041, 2943, 2862, 1675, 1558, 1431, 1384, 1200, 1173, 1128, 919 cm⁻¹; MS(EI)322(M⁺), 307(M⁺-CH₃), 280, 268, 251, 234, 207. Anal. Calcd.: C, 56.21; H, 4.69; N, 4.34. Found: C, 56.03; H, 4.79; N, 4.21.

2,3-Diacetoxymethyl-5-methoxyindol-4,7-dione (1d): Mp: 179-181° C.

TLC(dichloromethane: MeOH 98:2), R_{ƒ}=0.50. ¹HNMR(CDCl₃) δ9.74(1H, bs, indole proton), 5.72(1H, s, 6-proton), 5.79 and 5.24(2H, d, J=165.6 Hz, 3-methylene protons), 5.20(2H, s, 2-methylene protons), 3.84(3H, s, 5-methoxy), 2.11 and 2.04(6H, 2s, 2, 3-acetoxymethyls). ¹³C NMR(CDCl₃) δ56.47 ppm. IR(KBr pellet) 3443, 3371, 3342, 3260, 2993, 2918, 2827, 1674, 1634, 1569, 1404, 1374, 1241, 1139, 919, 827 cm⁻¹. MS(EI)322(M⁺), 280(M⁺-acetyl), 269, 251, 231, 216, 207, 179. Anal. Cal.: C, 56.21; H, 4.69; N, 4.34. Found: C, 56.18; H, 4.72; N, 4.33.

3-Acetoxy-7-methoxy-1,2,3,4-tetrahydrocyclopent[b]indole-5,8-dione(2b): MP: 173-175° C.

TLC: (dichloromethane: MeOH 95:5), R₇₁=0.55. ¹HNMR(CDCl₃) δ10.03(1H, bs, indole proton), 5.68(1H, s, 6-protons), 5.64(1H, m, 3-hydroxymethylene proton), 3.82(3H, s, 7-methoxy), 2.96, 2.73 and 2.25(4H, m, methylenes of cyclopentyl), 2.04(3H, s, 3-acetyl methyl). IR(KBrpellet)3452, 3206, 3035, 2949, 2892, 1701, 1623, 1577, 1502, 1433, 1322, 1219, 1167, 1024, 955, 834 cm⁻¹. MS(EI)275(M⁺), 247(M⁺-CO), 232, 214, 202, 187, 160, 131. Anal. Cal.: C, 61.08; H, 4.76; N, 5.09. Found: C, 60.94; H, 4.81; N, 5.00.

5-Methoxy-l-phenylsulfonylindole (3)

To 2.5 g (17 mmol) of 5-methoxyindole in 13 mL of dry THF, cooled by dry ice/acetone bath over a nitrogen atmosphere, was added 12 mL of 1.6 M n-butyllithium in hexane over 10 minutes. The reaction solution was stirred for 1.2 hours at 0° C. and then chilled in a dry ice acetone bath. To this chilled reaction was added 2.6 mL of PhSO₂Cl over 15 minutes and the resulting mixture stirred for 12 hours at room temperature. The reaction mixture was then mixed with 50 mL of 3% NaHCO₃ and stirred for 30 minutes. The solution was then extracted 4× with 100 mL portions of CH₂Cl₂. The extracts were dried over Na₂SO₄ and vacuum dried to a yellow oil. The oil was then mixed with 4 mL of THF followed by addition of 10 mL of hexane and kept at −10° C. for 5 hours. The crystallized product was then filtered and collected: 3.99g (82%) yield; MP 54-56° C.; TLC (CH₂Cl₂) R_{ƒ}=0.75; IR (KBr pellet) 3342, 3211, 3017, 2939, 2902, 2878, 1622, 1547, 1301, 1187, 1125, 1003, 842 cm⁻¹; ¹HNMR (CDCl₃) δ7.86, 7.52, 7.45, 6.93, and 6.59(10H, m, aromatic protons), 3.80(3H, s, 5-methoxy); MS (El mode) m/z 287(M⁺), 272(M⁺-CH₃), 147. Anal. Calcd (C₁₅H₁₃NO₃S): C, 62.71; H, 4.56; N, 4.88. Found: C, 62.43; H, 4.71; N, 4.80.

2-Formyl-5-methoxy-1-phenylsulphonylindole (4)

To 1.44g of 3 in 10 mL of THF, cooled at −78° C. under a nitrogen atmosphere, was added 4 mL of 1.6 M n-butyllithium in hexane over 10 minutes. The reaction solution was stirred for 25 minutes followed by addition of 0.7 mL ¹³C-DMF and stirring at 0° C. for 30 minutes. The completed reaction was mixed with 100 mL of saturated NH₄Cl solution and extracted 5×100 mL portion of CH₂Cl₂. The extracts were dried over Na₂SO₄ and dried to brown solid, which was further purifed by flash chromatography employing dichloromethane as the eluant. Yield: 1.21g (76%); MP 123-124° C.; TLC (dichloromethane), R_{ƒ}=0.44; ¹HNMR(CDCl₃) δ10.82 and 10.19(1H, d, J=189 Hz, 2-formyl protons), 8.13, 7.73, 7.53, 7.39, 7.13, and 6.99(9H, m, aromatic protons), 3.82(3H, s, 5-methoxy); 13C NMR(CDCl₃) δ182.7; IR (KBr pellet) 3354, 3326, 2917, 2889, 1679, 1532, 1514, 1347, 1236, 1218, 1036, 945, 834 cm⁻¹; MS(EI)316(M⁺), 301(M⁺-CH₃), 287(M⁺-¹³CO), 274, 257, 172, 146. Anal. Calcd. (C₁₆H₁₃N O₄S): C, 61.07; H, 4.14; N, 4.43. Found: C, 60.94; H, 4.20; N, 4.39.

2-Acetoxymethyl-3-formyl-5-methoxyindole (5)

To a solution of 200 mg Na metal in 10 mL of NH₃, cooled at −78° C., was added 630 mg of 4 dissolved in 10 mL of dry THF. The resulting solution was stirred for 15 minutes and then the reaction allowed to warm to room temperature after the addition of 0.5 g NH₄Cl. After the liquid NH₃ was gone, the solution was mixed with 20 mL of methanol containing 300 mg of NaBH₄. The solution was stirred for 10 minutes, diluted with 30 mL of water, and then extracted 4× with 100 mL portions of methylene chloride. The extracts were dried over Na₂SO₄ and evaporated to a residue. The residue was dissolved in 10 mL of methylene chloride containing 100 mg of dimethylaminopyridine. To this solution was added 300 λ acetic anhydride. The solution was stirred for 10 minutes and directly purified by a flash chromatography using methylene chloride as the eluent. The product was dissolved in 10 mL of methylene chloride and this solution was added to a solution of 200 λ POCl₃ and 600 λ DMF in 10 mL of methylene chloride at 0° C. The solution was stirred overnight and neutralized with saturated NaHCO₃ followed by extraction 6× with 50 mL portions of methylene chloride. The extract was dried over Na₂SO₄ and concentrated to a residue, which was further purifed by flash chromatography using methylene chloride as the eluent: Yield 123 mg(25%); Mp 189-192° C.; TLC(dichloromethane, methanol 98:2), R_{ƒ}=68; ¹HNMR(CDCl₃) δ10.27(1H, s, 3-formyl proton), 9.00(1H, bs, indole proton), 7.74(1H, d, J=2.7 Hz, 4-proton), 7.29(1H, d, J=8.7 Hz, 7-proton), 6.95(1H, dd, J=2.7 and J=8.7 Hz, 6-proton), 5.79 and 5.29(2H, d, J=150.9 Hz, 2-acetoxylmethyl), 3.89(3H, s, 5-methoxy), 2.16(3H, s, 2-acetoxylmethyl). ¹³C NMR(CDCl₃) δ56.10 ppm; IR(KBr pellet) 3494, 3337, 3169, 3088, 2962, 2812, 1665, 1616, 1537, 1518, 1198, 1174, 1102, 1031 cm⁻¹; MS(EI)248(M⁺), 206(M⁺-COCH₂), 188, 172, 159, 145, 116. Anal. Calcd. (C₁₃H₁₃NO₄): C, 63.30; H, 5.28; N, 5.64. Found: C, 62.99; H, 5.39; N, 5.52.

2-Acetoxymethyl-3-formyl-5-methoxy-4-nitroindole (6)

To a solution of 80 mg of 5 in 10 ml CH₂Cl₂ was added 50 λ of 70% HNO₃. The reaction was stirred for 0.5 hours and made basic with NaHCO₃ saturated solution. The basic solution was then extracted 4× with 40 mL portions of methylene chloride. The extracts were dried over Na₂SO₄ and dried, which was further purifed by flash chromatography. Yield: quantitative. Mp: 201-203° C. TLC(dichloromethane: MeOH 98:2), R_{ƒ}=0.58. ¹HNMR(CDCl₃) δ 10.04(1H, s, 3-formyl proton), 9.29(1H, bs, indole proton), 7.51 and 7.09(2H, 2d, J=8.7 Hz, 6, 7-protons), 5.88 and 5.38(2H, d, J=152.4 Hz, 2-acetoxylmethyl), 3.96 (3H, s, 5-methoxy), 2.19(3H, s, 2-acetoxylmethyl). ¹³C NMR(CDCl₃) δ 57.53 ppm. IR(KBr pellet) 3467, 3323, 3198, 3001, 2983, 2822, 1701, 1643, 1502, 1471, 1304, 1229, 1137, 1039, 927, 864 cm⁻¹. MS(EI)293(M⁺), 251(M⁺-COCH₂), 233, 215, 201, 185, 172, 156, 146, 116. Anal. Cal. (C₁₃H₁₂N₂O₆) C, 53.58; H, 4.12; N, 9.55. Found: C, 52.99; H, 4.28; N, 9.32.

¹³C-NMR Chemical Shifts (δ) of Labeled Products

2α-¹³C-1a, 56.28 ppm; 3α-¹³C-1a, 55.98 ppm; 3α-¹³C-1b, 55.5 ppm; 3α-¹³C-1c, 55.5 ppm; 3α-¹³C-1d, 56.5 ppm.

13C-NMR Study for the Eneimine Formation

A solution of 2 mg of 3α-¹³C-1c in 0.5 mL d₆-DMSO, containing 2 mg of 5% Pd on carbon, was then mixed with 0.5 mL of 0.1M pD=7.2 phosphate D₂O buffer. The solution was degassed with argon for 5 min followed by purging with H₂ until the quinone color of the solution disappeared. The solution was then degassed again with argon for 5 min, and the catalyst was removed by centrifuge in a nitrogen box. The supernatant was then transferred into a 3 mm NMR tube for the ¹³C-NMR spectrum obtained on a 100 MHz instrument after 4 h of scanning.

Hydrolysis of 3α-¹³C-1c

To a solution of 80 mg SM in 50 ml MeOH was added 60 mg 5% Pd on carbon. The solution was degassed with argon for 5 min, followed by passing H₂ through for 3 minutes. The solution was then degassed with argon for another 5 min and opened to air. The catalyst was filtered off through Celite and the filtrate was dried. The solid residue was purified by preparative TLC plate employing different eluents. The following products were isolated and characterized.

Ethyl 5-Methoxy-3-methylindole-4,7-dione -2-carboxylate (10)

Yield 2.36 mg (3.59%); TLC (CH₂Cl₂), R_{7ƒ}=0.62; ¹H NMR(CDCl₃) δ 9.61(1H, bs, indole nitrogen proton), 5.79(1H, s, 6-proton), 4.39(2H, q, J=7.2 Hz, 2-ethyl methylene), 3.86(3H, s, 5-methoxy methyl), 2.63(3H, d, J=129.6 Hz, 3-methyl), 1.40(3H, t, J=7.2 Hz, 2-ethyl methyl); ¹³C NMR(CDCl₃) δ 610.99; MS(EI) 264(M⁺), 245, 234, 217, 202.

Methyl 5-Methoxy-3-methylindole-4,7-dione -2-carboxylate (11)

Yield 0.3 mg (0.52%); TLC (CH₂Cl₂), R_{ƒ}=0.61; ¹H NMR(CDCl₃) δ 9.61(1H, bs, indole nitrogen proton), 5.79(1H, s, 6-proton), 3.86 and 3.76(6H, 2s, 2,5-methyls), 2.61(3H, d, J=129.6 Hz, 3-methyl); ¹³C NMR(CDCl₃) δ 11.21; MS(EI) 250(M⁺), 234, 217, 202.

Ethyl 5-Methoxy-3-methoxymethyl-lindole-4,7-dione -2-carboxylate (12)

Yield 1.73 mg (2.4%); TLC (CH₂Cl₂), R_{ƒ}=0.56; ¹H NMR(CDCl₃) δ 9.61(1H, bs, indole nitrogen proton), 5.78(1H, s, 6-proton), 4.76(2H, d, J=132.3 Hz, 3-methylene), 4.37(2H, q, J=7.5 Hz, 2-ethyl methylene), 3.86(3H, s, 5-methoxy methyl), 3.82(3H, d, J=6.6 Hz, 3-methoxy methyl), 1.40(3H, t, J=7.5 Hz, 2-ethyl methyl); ¹³C NMR(CDCl₃) δ 64.73; MS(EI) 294(M⁺), 262, 250, 233, 202.

Ethyl 3-(Ethyl 3′-methyl-indol-4′, 7′-dione-2′-carboxylate-5′-yl)methyl-5-methoxy-indol-4,7-dione-2-carboxylate (13)

Yield 2.37 mg (4.00%); TLC (CH₂Cl₂: acetone 98:2), R_{ƒ}=0.73; ¹H NMR(CDCl₃) δ 9.83 and 9.58(2H, 2bs, indole nitrogen protons), 5.96(1H, t, J=1.2 Hz, 6′-proton), 5.83(1H, s, 6-proton), 4.38(4H, m, methylenes of ethyls), 4.35(2H, doublet of triplet, J₁=1.2 Hz, J₂=132.8 Hz, 3-methylene), 3.84(3H, s, 5-methoxy methyl), 2.65(3H, d, J=129.6 Hz, 3′-methyl), 1.40 and 1.31(6H, m, methyls of ethyls); ¹³C NMR(CDCl₃) δ 24.58 and 10.90; MS(EI) 496(M⁺).

Ethyl 3-(Ethyl 5′-Methoxy-3′-methyl-indole-4′-one-7′-hydroxy-7′-ly)methyl-5-methoxy-indol-4,7-dione-2-carboxylate (14)

Yield 5.28 mg (7.73%); TLC (CH₂Cl₂: acetone 95:5), R_{ƒ}=0.70; ¹H NMR(CDCl₃) δ 9.99 and 9.53(2H, 2bs, indole nitrogen protons), 5.84(1H, s, 6′-proton), 5.53(1H, s, 6-proton), 4.28 and 4.16(4H, m, methylenes of ethyls), 3.87(6H, s, 5 and 5′-methoxy methyls), 3.74(2H, dd, J₁=12.8 Hz, J₂=133.6 Hz, 3′-methylene), 1.69(3H, d, J=124 Hz, 3′-methyl), 1.33 and 1.21(6H, m, methyls of ethyls); ¹³C NMR(CDCl₁₃) δ 38.38 and 10.56; MS(EI) 528(M⁺); Anal. Calcd. (C₂₆H₂₆N₂O₁₀): C, 59.09; H, 5.31; N, 5.33. Found: C, 58.68; H, 5.46; N, 5.12.

Ethyl 3-(Ethyl 5′-Methoxy-3′-acetoxymethyl-indole-4′-one-7′-hydroxy-7′-ly)methyl-5-methoxy-indol-4,7-dione-2-carboxylate (15)

Yield 7.03 mg (10.0%); TLC (CH₂Cl₂: acetone 95:5), R_{ƒ}=0.68; ¹H NMR(CDCl₃) δ 9.88 and 9.74(2H, 2bs, indole nitrogen protons), 5.85(1H, s, 6-proton), 5.67(1H, s, 6′-proton), 4.72(2H, dd,, J₁=6.8 Hz, J₂=144.8 Hz, 3-methylene), 4.30 and 4.14(4H, m, methylenes of ethyls), 3.87(6H, s, 5 and 5′-methoxy methyls), 3.85(2H, dd, J₁=12.8 Hz, J₂=164.8 Hz, 3′-methylene), 1.92(3H, s, 3-acetate methyl), 1.32 and 1.22(6H, m, methyls of ethyls); ¹³C NMR(CDCl₃) δ 56.64 and 38.53; MS(EI) 584(M⁺); Anal. Calcd. (C₂₈H₂₈N₂O₁₂): C, 57.53; H, 4.83; N, 4.79. Found: C, 57.17; H, 5.02; N, 4.68.

To a solution of 80 mg SM in 50 ml MeOH was added 60 mg 5% Pd on carbon. The solution was degassed with argon for 5 minutes, followed by passing H₂ through for 3 minutes. The solution was then degassed with argon for another 5 minutes and opened to air. The catalyst was filtered off through Celite and the filtrate was dried. The solid residue was purified by preparative TLC plate employing different eluents. The following products were isolated and characterized. (Reaction in MeOH with NaBH₄ as the reductant affords the same hydrolysis products).

2,3-Dimethyl-5-methoxy-indol-4,7-dione (16)

Yield 1.16 mg (2.27%); TLC (CH₂Cl₂: acetone 98:2), R_{ƒ}=0.70; ¹H NMR(CDCl₃) δ 9.37(1H, bs, indole nitrogen proton), 5.61(1H, s, 6-proton), 3.81(3H, s, 5-methoxy methyl), 2.24(3H, d, J=128.1 Hz, 3-methyl), 2.23(3H, s, 2-methyl); ¹³C NMR(CDCl₃) δ 9.69; MS(EI) 206(M⁺), 191.

2-Acetoxy-5-methoxy-3-methylindol-4,7-dione (17)

Yield 1.10 mg (1.68%); TLC (CH₂Cl₂: acetone 98:2), R_{ƒ}=0.68; ¹H NMR(CDCl₃) δ 9.42(1H, bs, indole nitrogen proton), 5.69(1H, s, 6-proton), 5.05(2H, s, 2-methyl), 3.82(3H, s, 5-methoxy methyl), 2.35(3H, d, J=128.7 Hz, 3-methyl), 2.09(3H, s, 2-acetate methyl);¹³C NMR(CDCl₃) δ 9.58; MS(EI) 264(M⁺), 249, 209, 202, 149.

2-Acetoxy-5-methoxy-3-methoxymethylindol-4,7-dione (18)

Yield 3.16 mg (4.33%); TLC (CH₂Cl₂: acetone 98:2), R_{ƒ}=0.65; ¹H NMR(CDCl₃) 69.63(1H, bs, indole nitrogen proton), 5.71(1H, s, 6-proton), 5.20(2H, s, 2-methylene), 4.71(2H, d, J=143.7 Hz, 3-methylene), 3.83(3H, s, 5-methoxy methyl), 3.42(3H, d, J=5.7 Hz, 3-methoxy methyl), 2.02(3H, s, 2-acetate methyl); ¹³C NMR(CDCl₃) 664.57; MS(EI) 294(M⁺), 279, 261, 250, 233, 218, 204, 190; Anal. Calcd. (C₁₄H₁₅NO₆): C, 57.13; H, 5.14; N, 4.76. Found: C, 56.97; H, 5.32; N, 4.73.

2-Acetoxymethyl-3-(2′-Acetoxymethyl-5′-methoxy-3′-methyl-5′,6′-dihydroindol-4′,7′-dione-5′-yl)methyl-5-methoxy-indol-4,7-dione (19)

Yield 0.75 mg (1.15%); TLC (CH₂Cl₂: acetone 95:5), R_{ƒ}=0.56; ¹H NMR(CDCl₃) δ 9.54, 9.19(2H, 2bs, indole nitrogen protons), 5.76(1H, s, 6-proton), 4.94(2H, m, 2-methylene), 4.52(2H, m, 2′-methylene), 3.87(3H, s, 5-methoxy methyl), 3.85 and 3.02(2H, m, 3-methylene), 3.63(3H, s, 5′-methoxy methyl), 3.62 and 2.64(2H, m, 6′-protons), 2.06 and 2.02(6H, 2s, acetate methyls), 1.78(3H, d, J=128.1 Hz, 3′-methyl); ¹³C NMR(CDCl₁₃) δ 29.36, 8.03; MS(EI) 528(M⁺).

2-Acetoxymethyl-3-(2′,3 ′-Diacetoxymethyl-5′-methoxy-5′,6′-dihydroindol-4′,7′-dione-5′-yl)methyl-5-methoxy-indol-4,7-dione (20)

Yield 1.03 mg (1.42%); TLC (CH₂Cl₂: acetone 95:5), R_{ƒ}=0.54; ¹H NMR(CDCl₃) δ 9.49, 9.31(2H, 2bs, indole nitrogen protons), 5.72(1H, s, 6-proton), 5.01(2H, m, 2-methylene), 4.70(2H, m, 2′-methylene), 4.28(2H, m, 3′-methylene), 3.84(3H, s, 5-methoxy methyl), 3.85 and 3.08(2H, m, 3-methylene), 3.63(3H, s, 5′-methoxy methyl), 3.61 and 2.72(2H, m, 6′-protons), 2.07, 2.04 and 1.97(9H, 3s, acetate methyls); ¹³C NMR(CDCl₃) δ 56.94, 29.36; MS(EI) 586(M⁺).

2-Acetoxymethyl-3-(2′-Acetoxymethyl-3′-(2″,3″-Diacetoxymethyl-5″-methoxy-indole-4″-one-7″-hydroxy-7″-ly-)methyl-5′-methoxy-indole-4′-one-7′-hydroxy-7′-ly-)methyl-5-methoxy-indol-4,7-dione (21)

Yield 4.28 mg (6.27%); TLC(CH₂Cl₂: acetone 90:10), R_{ƒ}=0.32; ¹H NMR(CDCl₃) δ 10.37, 9.96 and 9.75(3H, 3bs, indole nitrogen protons), 5.77(1H, s, 6″-proton), 5.55 and 5.34(2H, 2s, 6, 6′-protons), 5.60 and 5.40(2H, 2bs, 7, 7′-hydroxy protons), 5.06(2H, m, 2′-methylene), 4.54(2H, d, J=93.6 Hz, 3-methylene), 4.41(2H, m, 2″-methylene), 3.89, 3.84 and 3.65(9H, 3s, 5, 5′ and 5″-methoxy methyls), 4.12, 3.92, 3.74, 3.00 and 2.59(4H, m, 3′ and 3″-methylenes), 2.06, 2.01, 1.97 and 1.93(12H, 4s, acetate methyls); ¹³C NMR(CDCl₃) δ 56.84, 39.52 and 37.99; MS(EI) 828(M⁺). Anal. Calcd. (C₃₉H₄₁N₃O₁₇): C, 56.52; H, 5.34; N, 5.10. Found: C, 56.31; H, 5.52; N, 5.02.

2-Acetoxymethyl-3-(2′-Acetoxymethyl-5′-methoxy-3′-methyl-indole-4′-one-7′-hydroxy-7′-ly)methyl-5-methoxy-indol-4,7-dione (22)

Yield 0.78 mg (1.20%); TLC (CH₂Cl₂: acetone 90: 10), R_{ƒ}=0.53; ¹H NMR(CDCl₃) δ 9.57, 9.22(2H, 2bs, indole nitrogen protons), 5.74(1H, s, 6′-proton), 5.42(1H, s, 6-proton), 4.99(2H, m, 2′-methylene), 4.36(2H, 2m, 2-methylene), 3.87 and 3.83(6H, 2s, 5,5′-methoxy methyls), 3.82, 3.62, 3.18 and 2.75(2H, m, 3′-methylene), 2.07 and 2.03(6H, 2s, acetate methyls), 1.72(3H, d, J=126 Hz, 3-methy); ¹³C NMR(CDCl₃) δ 38.46, 8.87; MS(EI) 528(M⁺).

2-Acetoxymethyl-3-(2′-Acetoxymethyl-5′-methoxy-3′-methoxymethyl-indole-4′-one-7′-hydroxy-7′-ly-)methyl-5-methoxy-indol-4,7-dione (23)

Yield 8.84 mg (12.2%); TLC(CH₂Cl₂: acetone 90:10), R_{ƒ}=0.52; ¹H NMR(CDCl₃) 69.59 and 9.48(2H, 2bs, indole nitrogen protons), 5.73(1H, s, 6′-proton), 5.43(1H, s, 6-proton), 5.10(2H, m, 2′-methylene), 4.66(2H, d, J=147.0 Hz, 3-methylene), 4.56(2H, m, 2-methylene), 3.85 and 3.81(6H, 2s, 5 and 5′-methoxy methyls), 3.98, 3.54, 3.29 and 2.89(2H, m, 3′-methylene), 2.07, 2.04 and 1.99(9H, 3s, acetate methyls); ¹³C NMR(CDCl₃) δ 57.02 and 38.58; MS(EI) 586(M⁺); Anal. Calcd. (C₂₈H₂₈N₂O₁₂): C, 57.33; H, 4.81; N, 4.80. Found: C, 57.18; H, 4.87; N, 4.69.

2-Acetoxymethyl-3-(2′,3′-Diacetoxymethyl-5′-methoxy-indole-4′-one-7′-hydroxy-7′-ly-)methyl-5-methoxy-indol-4,7-dione (24)

Yield 0.65 mg (1.00%); TLC(CH₂Cl₂: acetone 90:10), R_{ƒ}=0.51; ¹H NMR(CDCl₃) δ 9.78 and 9.60(2H, 2bs, indole nitrogen protons), 5.70(1H, s, 6′-proton), 5.41(1H, s, 6-proton), 5.09(2H, m, 2′-methylene), 4.58(2H, m, 2-methylene), 4.46(2H, d, J=92.4 Hz, 3-methylene), 3.84 and 3.79(6H, 2s, 5 and 5′-methoxy methyls), 3.89, 3.54, and 3.00(2H, m, 3′-methylene), 3.23(3H, d, J=5.7 Hz, 3-methoxy methyl), 2.07 and 2.04(6H, 2s, acetate methyls); ¹³C NMR(CDCl₃) δ 65.05 and 38.15; MS(EI) 558(M⁺).

5′-Hydroxy-7,3′,7′-trimethoxy-1,2,3,4,2′,3′,4′,5′-octahydro-1′H-[3,5′]bi[cyclopent[b]indolyl]-5,8,8′-trione (25a)

TLC (CH₂Cl₂) R_{ƒ}=0.55; ¹HNMR(CDCl₃) δ 9.47 and 9.04 (2H, 2bs, 4 and 4′ protons), 5.65 and 5.46(2H, 2s, 6 and 6′ protons), 4.77(1H, m, 3′-proton), 3.87(1H, m, 3-proton), 3.85, 3.48, and 3.38(9H, 3s, 7, 3′, and 7′ methoxys), 2.89, 2.75, 2.47, 2.28, 2.09, 1.88, and 0.87(8H, mm, 1,2,1′, and 2′ protons).

3 ′-Acetoxy-5′-hydroxy-7,7′-dimethoxy-1,2,3,4,2′,3′,4′,5′-octahydro-1′H-[3,5′]bi[cyclopent[b]indolyl]-5,8,8′-trione (25b)

TLC (CH₂Cl₂) R_{ƒ}=0.52; ¹HNMR(CDCl₃) 9.66 and 9.40(2H, 2bs, 4 and 4′ protons), 5.61 and 5.40(2H, 2s, 6 and 6′ protons), 4.60(1H, m, 3′-proton), 3.84(1H, m, 3-proton), 3.85 and 3.34(6H, 2s, 7 and 7′ methoxys), 3.59(1H, bs, 5′-hydroxy proton), 2.61, 2.45, 2.01, 1.73, 1.10, and 0.87(8H, mm, 1, 2, 1′, and 2′ protons).

Preparation of the ¹³C-labeled indoloquinone-DNA adduct

The DNA hexamer d(ATCGAT)₂ was prepared by the phosphorimidate method and purified by 20% preparative polyacrylamide gel. The DNA adducts were prepared by mixing 8.0 mg (2.0 μmol of strand) of d(ATCGAT)₂ in 0.05 M of tris buffer (pH 7.4) with 1.0 mg (3.0 μmol) of the quinone in 0.25 mL of DMSO. To the mixture was added 0.2 mg of 5% Pd on Carbon. The resulting mixture was degassed under argon for 30 minutes and followed by purging with H₂ at 1 atm for 20 minutes. The mixture was then purged with argon for 10 minutes and incubated at 30° C. for 2 h. The reaction was opened to the air and the catalyst was centrifuged off. To the supernatant was added 0.45 mL of 7.5 M ammonium acetate, 10 mL of cold ethanol and left in a −20 ° C. freezer for 24 h. The mixture was centrifuged at 12000g for 20 minutes and the supernatant was removed. The DNA pellet was redissolved in water, ethanol precipitated again and dried. The pellet was then dissolved in D₂O and lyophilized twice before diluting in 0.7 mL 100 % D₂O.

Materials for Sequencing Studies

Quinone stock solutions were made up in DMSO at 10 mM, 1 mM, and 0.1 mM. Electrophoresis-grade acryalmide and bis(acrylamide) were purchased from Sigma, ultrapure urea and agarose from GIBCO-BRL, and piperidine from Sigma. The pBR322 plasmid DNA, restriction enzymes EcoRI and RsaI, T4 polynucleotide kinase (PNK), and bacterial alkaline phosphatase (BAP) were purchased from Gibco BRL Life Technologies. The 5′-[γ-³²P]ATP were purchased from New England Nuclear Research Products. For sequencing labeled DNA, ³²P-end labeled 514 base pair restriction fragments were prepared by first digesting supercoiled pBR322 plasmid DNA with EcorR I restriction endonuclease. 5′-end labeling was achieved by the treatment of the EcorR I pre-cleaved DNA with alkaline phosphatase, [γ-³²P]ATP and T4 polynucleotide kinase. Following the end-labeling, the DNA was further digested with RsaI to yield the 514 base pair fragment that was purified by 6% preparative non-denaturing gel electrophoresis with TBE buffer (TBE electrophoresis buffer is 90 mM Tris, 90 mM boric acid, and 2 mM EDTA at pH 8.3).

Cleavage of ³²P-end labeled DNA

Indoloquinone-induced cleavage reactions were carried out in two different reaction conditions. For catalytic reduction, the reaction was carried out in 200 μL total volumes containing calf thymus DNA (500 μM nucleotide concentration) 2×10⁴ cpm ³²P-end labeled restriction fragment and 100 μM of drug in DMSO in 25 mM phosphate buffer (pH 7.4) with 0.05 mg 5% Pd on carbon as catalyst. Reaction mixtures were degassed under argon for 5 min before being initiated by the bubbling of H₂ at 1 atm for 30 seconds. The reaction was then degassed under argon for 5 minutes and incubated at 37° C. for 30 minutes. For sodium boromhydride reduction, the reaction was carried out in 200 μL total volumes containing calf thymus DNA (500 μM nucleotide concentration) 2×10⁴ cpm ³²P-end labeled restriction fragment and 100 μM of drug in DMSO in 25 mM phosphate buffer (pH 7.4). Reaction mixtures were degassed under argon for 5 minutes before being initiated by the addition of 100 mM sodium boromhydride in water. The reaction incubated at 37° C. for 30 minutes. Under both reaction condition, the reactions were quenched by ethanol precipitation in the presence of 0.3 M sodium acetate. The DNA pellet was washed with 70% ethanol, dried, and resuspended in 3 μL of 80% formamide loading dye. Piperidine treatment was also carried out by dissolving the DNA pellet in 20 μL of fresh 0.2 M piperidine, heated at 90° C. for 10 minutes and lyophilized. The samples, along with the Maxam-Gilbert A+G sequencing reaction mixtures, were then loaded onto 12% polyacrylamide/7.5 M urea denaturing gels and electrophoresed at 1200 V for 5 h in the TBE buffer described above. Autoradiography of the gel was conducted at −70° C. using Kodak X-omat film.

Modeling into DNA

INSIGHT II from Molecular Simulations, Inc. (San Diego) was used for modeling studies and consistent valence force field (CVFF) was utilized for all minimization protocols.

The cyclopent[b]indole quinone methide and the B-DNA sequence were constructed utilizing fragments available in fragment libraries of BUILDER module and minimized utilizing consistent valence force field (500-1000 iterations). The quinone methide reaction center was contrained to within a bond length of either the guanine N(7) or thymine O(6) nucleophiles. The best-fit structure was achieved by using the DOCKING module of INSIGHT II employing the consistent valence forcefield (CVFF). The orientation with the lowest energy (van der Waals and electrostatic) was chosen as the optimal DNA-bound structure.

What is claimed is:

1. An eneimine dimer having the structural formula:

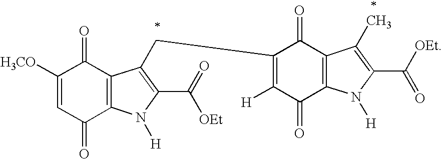

2. An eneimine dimer having the structural formulae: 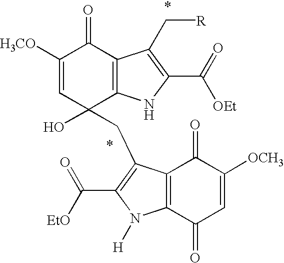
wherein R is H or OAc.

3. The eneimine dimer according to claim 2, in which R is Hydrogen.

4. The eneimine dimer according to claim 2, in which R is OAc.

5. An eneimine dimer having the structural formulae: 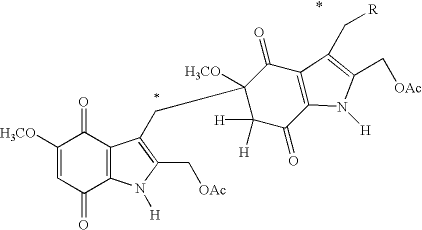
wherein R is H or OAc.

6. The eneimine dimer according to claim 5, in which R is H.

7. The eneimine dimer according to claim 5, in which R is OAc.

8. An eneimine trimer having the structural formula:

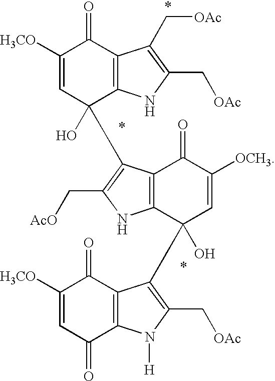

9. An eneimine dimer having the structural formulae: 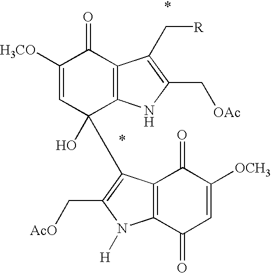
wherein R is H, OAc or OCH₃.

10. The eneimine dimer according to claim 9 wherein R is Hydrogen.

11. The eneimine dimer according to claim 9 wherein R is OAc.

12. The eneimine dimer according to claim 9 wherein R is OCH₃.

13. A method for incorporating a ¹³C label in the 2-α position of an indole suitably substituted with substituent R, comprising the following steps:

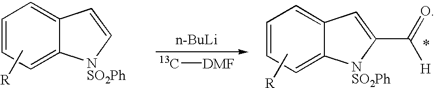

14. The method according to claim 13, comprising the following steps:

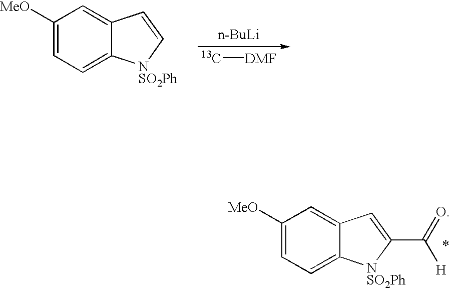

15. A method for crosslinking the G-base and phosphate backbone of DNA comprising subjecting DNA to the eneimine having the structural formula: 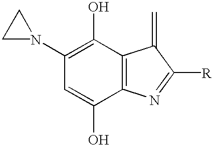
wherein R is any suitable substituent.

16. The method according to claim 15 wherein R is an ethoxy carbonyl, hydroxymethyl or acetoxy methyl.

17. The method according to claim 16, wherein R is ethoxy carbonyl.

18. The method according to claim 16, wherein R is hydroxymethyl.

19. The method according to claim 16, wherein R is acetoxy methyl.

20. A method for intercalating and alkylating DNA comprising subjecting DNA to a cyclopent[b]indole quinone methide having the formula: 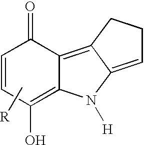
wherein R is any suitable substituent.

21. The method according to claim 20 comprising subjecting the DNA to cyclopent[b]indole quinone methide.

22. A method for the recognition of 3′-GT′5′ and 3′-GGA-5′ DNA sequences comprising subjecting DNA to the enemine dimer according to claim 1.

23. A method for the recognition of 3′-GT′5′ and 3′-GGA-5′ DNA sequences comprising subjecting DNA to the enemine dimer according to claim 2.

24. A method for the recognition of 3′-GT′5′ and 3′-GGA-5′ DNA sequences comprising subjecting DNA to the enemine dimer according to claim 5.

25. A method for the recognition of 3′-GT′5′ and 3′-GGA-5′ DNA sequences comprising subjecting DNA to the enemine trimer according to claim 8.

26. A method for the recognition of 3′-GT′5′ and 3′-GGA-5′ DNA sequences comprising subjecting DNA to the enemine dimer according to claim 9.
